(19) United States
(12) Patent Application Publication
(10) Pub. No.: US 2025/0381055 A1
(43) Pub. Date: Dec. 18, 2025

(54) ANTI-REFLUX OSTOMY DEVICE

(71) Applicants: OWNMED INNOVATION S.L., MADRID (ES); Fundación para la investigación Biomédica del Hospital Gregorio Marañón (FIBHGM), Madrid (ES); Fundación Instituto de Investigación Sanitaria fundación Jiménez Díaz (FIIS-FJD), Madrid (ES); Universidad Carlos III De Madrid (UC3M), Leganés (Madrid) (ES)

(72) Inventors: Ana Ye Zhang, Madrid (ES); Manuel Desco Menéndez, Madrid (ES); Jesús García-Foncillas López, Madrid (ES)

(21) Appl. No.: 18/877,590

(22) PCT Filed: Jun. 22, 2023
(86) PCT No.: PCT/EP2023/067026

§ 371 Date: Dec. 20, 2024

(30) Foreign Application Priority Data
Jun. 22, 2022 (EP) 22382591.0

(51) Int. Cl. A61F 5/44; A61F 5/443; A61F 5/445

(52) U.S. Cl. CPC A61F 5/4405; A61F 5/443; A61F 5/445

(57) ABSTRACT

A stoma plug suitable for controlling the output of biological fluids from a colon, ileum and/or ureter through a stoma created by a colostomy, ileostomy or urostomy, respectively. The stoma plug includes a tube comprising an opening in a proximal portion and at least one opening in a distal portion, a two-way valve system including a base element and a plug, the two-way valve system connected to the tube through the base element, the two-way valve system configured to control the fluid flow through the opening of the proximal portion of the tube, and a cap configured to cover the two-way valve system and the stoma, the cap including an opening to directly access the two-way valve system from the outside. The tube is configured to be inserted into the colon, ileum and/or ureter through its distal portion, and the two-way valve system is configured to passively remain closed and to be opened when the plug is displaced from the at least one opening

### TECHNICAL FIELD OF THE INVENTION

The present invention pertains to the field of ostomy devices, particularly to a stoma plug for controlling the output of biological fluids through a stoma created by a colostomy, ileostomy or urostomy and to a system comprising such stoma plug and an element for managing the outputted biological fluids.

### BACKGROUND OF THE INVENTION

An ostomy is an opening in the abdominal wall through which the intestine is brought out to allow for the elimination of feces/urine due to a medical problem that prevents their elimination in a natural way, through the anus or urethra. A patient may require an ostomy for various reasons, such as colorectal cancer, inflammatory bowel diseases (IBD) such as Crohn's disease and ulcerative colitis, severe diverticulitis with peritonitis, severe trauma, birth defects, and spinal cord injury, among others.

A stoma may be temporary or permanent and may require a collection system (collecting bag) for fecal or urinary material. Currently, there are various types of collecting bags available, including open and closed bags, one-piece or two-piece bags, transparent or opaque, of different sizes and brands. The bag should be emptied or changed when it is one-third or half full. Closed bags are indicated for fecal material of hard consistency, while open bags are indicated for liquid fecal/urine material, which can be reused. With this type of bags, there is a possibility of getting in contact with feces every time the bag is emptied and closed again.

Ostomies can be categorized into three types: ileostomies, colostomies, and urostomies, which can be temporary or permanent. An ileostomy creates an opening from the ileum to the exterior of the body through the abdominal wall.

A colostomy creates an opening from the colon to the exterior of the body through the abdominal wall. Four types of colostomies can be identified depending on the part of the colon where the colostomy is performed: ascending, transverse, descending, or sigmoid.
The ascending colostomy is performed in the ascending part of the colon, starting at the cecum and going up the right side of the abdomen. The ascending colostomy is located in the right flank of the abdomen. The biological material is usually liquid to semi-liquid, and gas is expelled. This type of colostomy is rare and is not usually performed as doctors opt for an ileostomy.
The transverse colostomy is performed in the transverse part of the colon. The transverse colostomy is usually located in the center of the abdomen, above the navel. The biological material is usually of liquid to pasty consistency, and gas is frequent.
The descending colostomy is performed in the descending part of the colon. The descending colostomy is located in the lower left abdomen. The biological material varies from pasty consistency to formed consistency, and gas is frequent.
The sigmoid colostomy is performed in the sigmoid colon (part of the colon that connects to the rectum). The sigmoid colostomy is located in the lower left abdomen. The stool is of pasty to formed consistency, and gas is frequent. The sigmoid colostomy is the most common type of colostomy.

A urostomy creates an opening from the intestine (usually the ileum) to the exterior of the body through the abdominal wall, which allows the urine to exit the body. The ureters are separated from the bladder and reconnected to a part of the ileum (a portion of the ileum is cut, and the rest of the ileum is connected to the large intestine), allowing it to serve as a small reservoir for storing urine.

According to the International Agency for Research on Cancer (IARC), colorectal cancer is the third most prevalent cancer worldwide, with an incidence of 19.5% in 2020. In addition, colorectal cancer is the second most common cause of cancer death, causing nearly 1 million deaths per year, and is one of the main reasons (80%) for ostomy surgery in Spain. It is estimated that 1.5 out of every 1,000 Spanish citizens have an ostomy, which equates to 70,000 people, with over 13,000 new cases each year. The number of ileostomy procedures in Europe was 241,406 in 2019, and currently, 13.4 million people in the world have a stoma, of which 7.7 million people have ostomies with liquid waste.

The risk of colorectal cancer increases with age, as it is commonly diagnosed in people aged 65 to 74 years. However, a trend is being observed in which 25% of colon cancer patients are younger than 50 years. Additionally, the prevalence of colorectal cancer is increasing in Asian countries such as India, China, and Japan. According to estimates from the American Cancer Society, in 2020, the number of new cases of colon and rectal cancer in the United States was 104,610 and 43,340, respectively.

According to a 2019 report from the Crohn's and Colitis Foundation, approximately 1.6 million Americans currently have inflammatory bowel diseases, and 70,000 new cases of IBD are diagnosed each year in the United States. Likewise, according to a 2016 WHO report, of the global prevalence of ostomies, 42% are colostomies, 41% are ileostomies, and 16% are urostomies.

The growing elderly population is a key factor in the increasing ostomy care market. According to the WHO, the percentage of people over 65 years old is expected to increase from 7% in 2000 to 16% in 2050. Additionally, the majority of people with an ostomy are over 50 years old, and according to the American Medical Association, ostomy procedures are more common in older individuals. Furthermore, according to the NCBI, patients over 70 years old are more likely to undergo permanent ostomy procedures, with longer hospitalizations and a higher mortality rate.

As per Crohn's Colitis Foundation 2019 report, approximately 1.6 million Americans currently have Inflammatory Bowel Diseases (IBD) and 70,000 new cases of IBD are diagnosed each year in the U.S. Moreover, according to the WHO report 2016, the global prevalence of colostomies, ileostomies, and urostomies was 43.0%, 41.0%, and 18.0% respectively. According to NCBI, IBD comprises ulcerative colitis or Crohn's disease, and its occurrence in European countries is gradually rising. In Europe, around 731,000 people are living with an ostomy and about 0.3% of the total population suffers from IBD. Moreover, as per the Colostomy Association Ltd., trading as Colostomy U.K., 1 in every 500 people in the U.K. is an ostomate.

Currently, there are no self-control devices for ostomies with liquid or semi-liquid waste material, so ostomized patients may face physical, psychological, and economic implications, such as changes in their body image, continuous loss of stool or urine, odor, sexual dysfunction, anxiety, depression, and emotional concerns related to the stoma, the need for psychological preparation and support, and economic expenses to buy ostomy wafers and collection bags. The existing devices, as mentioned earlier, are collection bags, which do not provide the ostomized patient with control over their continence. The main function of these bags is to collect fecal/urinary material that is constantly and unconsciously expelled.

When the colostomy puts out stool at regular (expected times), it may be able to use a stoma cover instead of always wearing a pouch. Plastic, ready-made stoma caps are also available. However, for ileostomies and urostomies, where the stool is more liquid, the stoma caps do not provide an efficient solution that can avoid spills, especially when the pouch is to be attached.

There is therefore a need for an ostomy device that can provide self-control for stool continence, no need to change the anti-reflux cap every time the stool is removed (reusable), increased self-esteem for carriers, at a low cost and with a painless insertion of the device.

### SUMMARY OF THE INVENTION

A first aspect of the invention refers to a stoma plug suitable for controlling the output of biological fluids from a colon, ileum and/or ureter (50) through a stoma (45) created by a colostomy, ileostomy or urostomy respectively. The stoma plug comprises: a tube (10) comprising an opening (12) in a proximal portion and at least one opening (14) in a distal portion, a two-way valve system (20) comprising a base element (22) and a plug (24), the two-way valve system (20) connected to the tube (10) through the base element (22), the two-way valve system (20) configured to control the fluid flow through the opening (12) of the proximal portion of the tube (10) and a cap (30) configured to cover the two-way valve system (20) and the stoma (45), the cap (30) comprising an opening (32) to directly access the two-way valve system (20) from the outside. The tube (10) is configured to be inserted into the colon, ileum and/or ureter (45) through its distal portion. Preferably, the tube (10) is configured to be inserted into a stoma of the colon, ileum and/or ureter (45).

The two-way valve system (20) is selected from one of the following two alternatives:
i. the two-way valve system (20) further comprises an actuator (26), the base element (22) comprises at least one opening (21), wherein the two-way valve system (20) is configured to passively remain closed such that when the actuator (26) is not actuated the plug (24) is configured to cover the base element (22) and seal the at least one opening (21); and is further configured to be opened when the plug (24) is displaced from the at least one opening (21) such that the actuator (26) is configured to displace the plug (24) and unseal the at least one opening (21) of the two-way valve system (20) when actuated. The plug (24) is further configured to reseal the at least one opening (21) of the two-way valve system (20) when the actuator (26) stops being actuated; or
ii. the two-way valve system (20) further comprises a spring (27) and a plug stopper (29) comprising at least one opening (21), the base element (22) configured to be slidably receivable by the spring (27), wherein the two-way valve system (20) is configured to passively remain closed such that the spring (27) is configured to press the plug (24) against the plug stopper (29) and close the at least one opening (21) of the two-way valve system (20) and is further configured to be opened when the spring (27) is longitudinally displaced from the plug stopper (29) when a force is applied, thus displacing the plug (24) from the at least one opening (21) and therefore opening the at least one opening (21) of the two-way valve system (20).

In a preferred embodiment, the two-way valve system (20) is selected from the first alternative i). In an alternative preferred embodiment, the two-way valve system (20) is selected from the second alternative ii).

In a preferred embodiment wherein the two-way valve system (20) is according to the first alternative i), the base element (22) is configured to perforate the plug (24) when the plug (24) is displaced by the actuator (26). In a more preferred embodiment, the base element (22) has a spiky shape, more preferably wherein the spiky shape is essentially of a cone shape. In a preferred embodiment wherein the two-way valve system (20) is according to the second alternative ii), the plug (24) comprises two pieces (24A, 24B). The two pieces (24A, 24B) are configured to remain in contact when the spring (27) presses them against the plug stopper (29) and to separate apart when the spring (27) is longitudinally displaced thus generating an aperture between the two pieces (24A, 24B) and allowing the at least one opening (21) of the two-way valve system (20) to be accessible through the aperture generated between the two pieces (24A, 24B).

In another preferred embodiment of any of the previous embodiments, the tube (10) comprises a support element configured to receive the base element (22) of the two-way valve system (20), and wherein the two-way valve system (20) is comprised within the tube (10).

In a preferred embodiment of any of the previous embodiments, the at least one opening (14) on the distal portion of the tube (10) is at least laterally placed on the tube (10).

In another preferred embodiment of any of the previous embodiments, the at least one opening (14) on the distal portion of the tube (10) is smaller than 15 mm.

In another preferred embodiment of any of the previous embodiments, wherein the tube (10) comprises a rounded tip (15) on its distal portion, configured to facilitate the insertion of the tube (10) through the stoma (45). In a more preferred embodiment, the rounded tip (15) on the distal portion of the tube (10) has a rounded cone shape.

In another preferred embodiment of any of the previous embodiments, the tube (10) external surface further comprises at least a spiral portion configured to increase the contact surface of the tube (10) with the stoma (50).

In another preferred embodiment of any of the previous embodiments, the tube (10) is comprised of any of the materials selected from: medical grade silicone, surgical plastic thermoplastic elastomers or any other biocompatible material including biocompatible polymers.

In another preferred embodiment of any of the previous embodiments, the tube (10) further comprises a lubricated layer on its external surface, configured to reduce the friction with the stoma (45) during its insertion.

In another preferred embodiment of any of the previous embodiments, the two-way valve system (20) further comprises a heat-sensitive hydrocolloid surface or an adaptative biocompatible material surface configured to stick or adapt to the external stoma mucosa (45). In a further preferred embodiment, the heat-sensitive hydrocolloid or the adaptative biocompatible material surface comprises a dressing configured to adhere to the subject's skin (40) extending radially from the two-way valve system (20). In an even further preferred embodiment, the dressing is transparent.

In another preferred embodiment of any of the previous embodiments, the at least one opening (14) of the distal portion of the tube (10) is at least laterally placed on the tube (10).

A second aspect of the invention refers to a stoma plug system. The stoma plug system comprises a stoma plug according to any of the previous claims, and a collecting bag or a vacuum device. The collecting bag or vacuum device is configured to connect to the two-way valve system (20) and to open the at least one opening (21) by displacing the plug (24) from the at least one opening (21). In a preferred embodiment, the collecting bag or vacuum device opens the at least one opening (21) through its connection to the two-way valve system (20). In another preferred embodiment, the vacuum device is a syringe.

### BRIEF DESCRIPTION OF THE DRAWINGS

To enable better understanding of the present disclosure, and to show how the same may be carried into effect, reference will now be made, by way of example only, to the accompanying schematic drawings, in which:

FIG. 1 shows an exploded view of a stoma plug according to at least one of the embodiments of the invention.

FIG. 2 shows a perspective view of a stoma plug according to at least one of the embodiments of the invention.

FIG. 3 shows a schematic cross-sectional view of a stoma plug according to at least one of the embodiments of the invention.

FIG. 4 shows a schematic cross-sectional view of an actuated stoma plug according to at least one of the embodiments of the invention.

FIG. 5 shows another schematic cross-sectional view of a stoma plug according to at least one of the embodiments of the invention.

### DESCRIPTION OF THE INVENTION

### Definitions

The term “two-way valve system” is preferably understood as a valve system capable of allowing fluids to flow in both senses. The fluid flow sense will be determined by the pressures at each side of the valve, such that the flow will go from the side where the fluid pressure is higher to the side where the fluid is lower.

The term “vacuum device” is preferably understood as any device capable of creating a negative pressure. It may therefore be a vacuum connection from a hospital, a portable fluid vacuum device or simpler device such as a syringe.

### Description

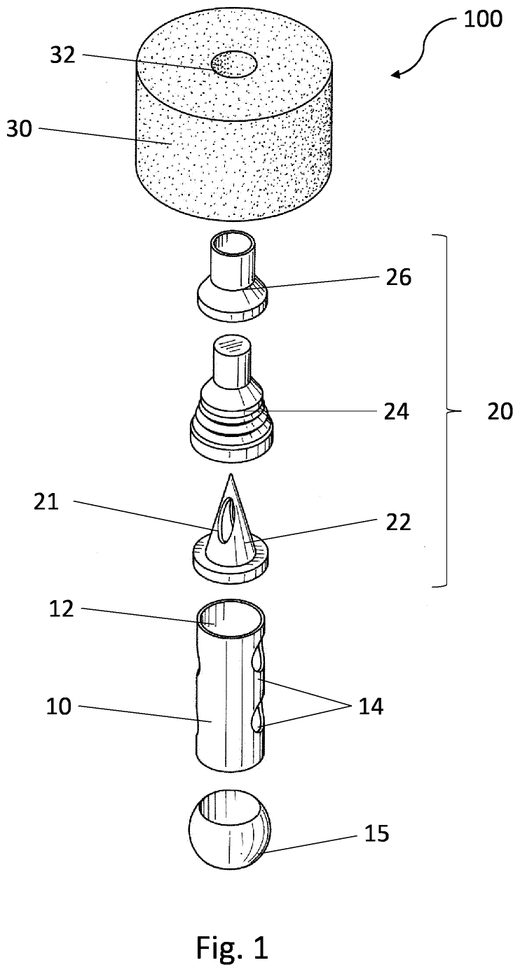

A first aspect of the present invention refers to a stoma plug suitable for controlling the output of biological fluids from a colon, ileum and/or ureter (50) through a stoma (45) created by a colostomy, ileostomy or urostomy. As shown with respect to FIG. 1, the stoma plug 100 comprises: a tube 10 comprising an opening 12 in a proximal portion and at least one opening 14 in a distal portion. It is noted that in FIG. 1 the tube 10 comprises two openings 14 in the distal portion but in other embodiments the tube may comprise only one opening 14 or more than two openings 14 in the distal portion. The stoma plug 100 further comprises a two-way valve system 20 comprising at least one opening 21 and a plug 24 configured to close the at least one opening 21. The two-way valve system 20 connected to the tube 10 through its proximal portion. It is noted that in FIG. 1 the two-way valve system 20 comprises other optional elements 22 and 26 but in other embodiments the plug may comprise only at least one opening 21 and a plug 24 configured to close the at least one opening 21. Lastly, the stoma plug 100 comprises a cap 30 configured to cover the two-way valve system 20 and the stoma 45. The cap 30 comprises an opening 32 to directly access the covered two-way valve system 20 from the outside.

The tube 10 is configured to be inserted into the colon, ileum and/or ureter 50 through its distal portion, and the two-way valve system 20 is configured to passively remain closed and to be opened when the plug 24 is displaced from the at least one opening 21.

Having a tube comprising an opening 12 in a proximal portion and at least one opening 14 in a distal portion advantageously allows it to fluidly connect the colon, ileum and/or ureter 50 biological fluids with the two-way valve system 20 through the at least one opening 14 of the distal portion. In use the tube 10 is inserted into the colon, ileum and/or ureter 50 through its distal portion and according to a preferred embodiment of the invention, the two-way valve system 20 is connected to the tube 10 through the proximal portion of the tube 10. Therefore, the biological fluids present at the colon, ileum and/or ureter 50 can pass through the opening 14 of the tube 10 and reach the opening 12 where the two-way valve system 20 is connected. Therefore, the two-way valve system 20 is configured to control the pass of the biological fluids through the stoma 45. Preferably, the two-way valve system 20 is configured to control the fluid flow through the opening 12 of the proximal portion of the tube 10.

The two-way valve system 20 may be connected to the tube 10 by different ways. Preferably, the two-way valve system 20 is connected to the tube through the base element 22. The base element 22 may be screwed to the tube 10, or glued to the tube 10. Alternatively, the base element 22 may be snaped in the tube 10. The person skilled in the art may foresee many other alternatives in which the base element and the tube 10 may be connected to each other.

It is noted that the two-way valve system 20 and the tube 10 may be connected in different locations. According to a preferred embodiment of the invention, the two-way valve system 20 is connected to the tube 10 through the proximal portion of the tube 10. According to another alternative preferred embodiment of the invention, the two-way valve system 20 is comprised within the tube 10. In this preferred embodiment, the two-way valve system 20 may be comprised within the tube 10 in its distal portion, in its proximal portion or anywhere between the proximal and the distal portion of the tube 10. It is further noted that in this preferred embodiment, the two-way valve system 20 may extend along the tube 10 towards the proximal portion of the tube 10, and may further extend out from the tube 10 through the opening 12 in the proximal portion of the tube 10.

The two-way valve system 20 advantageously allows it to provide a way to control the flow of fluids in both directions, the flow of the biological fluids from the colon, ileum and/or ureter 50 through the tube 10 towards the exterior, and the flow of any fluid from the exterior to the colon, ileum and/or ureter 50 through the tube 10. Therefore, it allows to control the passage of any fluids at the stoma level. It is noted that in FIG. 1 the two-way valve system 20 further comprises elements 22 and 26 but in other embodiments the same advantages can be achieved using other elements.

It is noted that the person skilled in the art may devise several ways of making a two-way valve system 20. Although the present invention mainly describes two alternative ways of such a two-way valve system 20, it is not limited to any particular type of two-way valve system 20 comprising at least one opening 21 and a plug 24 configured to close that plug passively and to be opened when the plug 24 is displaced form the at least one opening 21. Also, more complex systems comprising more than one plug 24 and not being configured to be passively closed but actively closed (either manually or mechanically) may be envisaged by the person skilled in the art.

The cap 30 advantageously covers both the two-way valve system 20 the stoma external mucosa 45 and the peristomal skin, so that external elements such as clothes do not interact with neither the two-way valve system 20 which could lead to an undesired opening of the valve nor the stoma external mucosa 45, which could lead to a pain source. The cap 30 comprises an opening 32 to access the two-way valve system 20 so that the fluids can be extracted and optionally to operate the valve, i.e. to open it when desired. It is noted that in although in FIG. 1 the opening 32 is centred in the cap surface, the opening 32 may be located in any part of the cap 30 surface. The cap may comprise different shapes in order to provide a slimmer and less noticeable device. It may also comprise different materials, for example elastic and/or flexible materials that can adapt to the user's skin surface 40 and/or the stoma external mucosa 45.

It is noted that when the two-way valve system 20 is comprised within the tube 10, the opening 32 cap 30 still provides an access to the two-way valve system 20. This is done through the opening 12 of the proximal portion of the tube 10 and through the tube itself. Therefore, the two-way valve system 20 can still be actuated from the exterior to open it and therefore allow the fluid flow through the opening (12) of the proximal portion of the tube (10),

It is also noted that the tube 10, the two-way valve system 20 and the cap 30 shapes and designs of FIG. 1 are only for illustrative purposes. Therefore, the tube 10 may have a section different to a circular one, or may be longer or shorter. The two-way valve system 20 may be more or less compact and have different ways of functioning. Also, the cap 30 may take different shapes other than cylindrical to cover the two-way valve system 20 and the stoma 45. Similarly, the different openings 12, 14, 21, 32 may take different shapes and the circular and oval ones in the drawings are for illustrative purposes. Therefore, different opening shapes and patterns may be used as the person skilled in the art will notice. For example, they may be shaped in form of an oval or ellipse, or when more than one opening of one type is present, they may be disposed in different configurations as to maximise the flow rate capacity and/or to reduce the fluid turbulence when a fluid flows through them.

FIG. 1 comprises an optional rounded tip 15 at the distal portion of the tube 10 that it may not be present in other embodiments. Also, the shape of such tip may have a different shape.

The two-way valve system 20 is configured to passively remain closed. Advantageously, this allows for the stoma plug to be configured to prevent the flow of the biological fluids when in use. Therefore, the stoma plug according to any embodiment of the first aspect of the invention provides an ostomy device that can provide self-control for stool continence as only when the plug 24 is removed from the opening 21 the biological fluids such as the stool can flow outwards from the colon, ileum and/or ureter 50 through the stoma 45. Moreover, as the two-way valve system 20 can be closed again once opened, there is no need to change the anti-reflux cap every time the stool is removed, making it reusable. Altogether, this provides an increased self-esteem for carriers, at a low cost. Moreover, as the tube 10 is configured to be inserted into the stoma, it provides all these advantages with a painless insertion of the device.

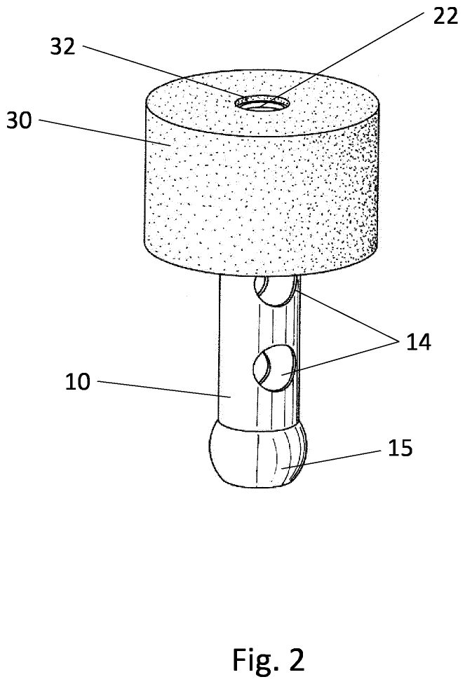

FIG. 2 shows a perspective view of a stoma plug according to at least one of the embodiments of the invention. It comprises a tube 10, a two-way valve system 20 (not seen) and a cap 30. It can be seen that the cap 30 covers the two-way valve system 20 and that the opening 32 of the cap 30 allows to directly access the two-way valve system 20 from the outside. It is noted that the tube 10 comprises more than one opening 14 in the distal portion, but these are optional and in other embodiments, the tube 10 may comprise only one or a different number of openings 14 in the distal portion of the tube 10. Moreover, the stoma plug of FIG. 2 further comprises an optional rounded tip 15 in the distal portion of the cap 10 and other embodiments may not comprise such rounded tip 15.

In a preferred embodiment, as shown in FIG. 1, the two-way valve system 20 further comprises: a base element 22 comprising the at least one opening 21 of the two-way valve system 20, and an actuator 26. The plug 24, is configured to cover the base element 22 and seal the at least one opening 21 when the actuator 26 is not actuated. Moreover, the actuator 26 is configured to displace the plug 24 and unseal the at least one opening 21 of the two-way valve system 20 when actuated. The actuator 26 may be actuated in different ways. For example, as shown in FIG. 1 the actuator 26 can longitudinally displace the plug 24 towards the base element 22, and allow the at least one opening 21 to be unsealed. However, the person skilled in the art may appreciate there are many ways an actuator can displace a plug 24 from an opening 21. For example the plug 24 may be comprised of a plurality of parts that are configured to laterally displace when the plug is longitudinally displaced, therefore displacing form the opening 21. Alternatively, the plug 24 may simply be shaped such that when it is longitudinally displaced, the opening 21 is no longer covered by the seal 24. For example, this may be done through a particular design of the plug 24, or through rotating means configured to rotate the plug 24 as it is longitudinally displaced. According to another exemplary embodiment of this preferred embodiment, the plug 24 is made of an elastic material, such as rubber that can be perforated by the base element 22 to unseal the opening 21, as shown with reference to FIG. 1.

Moreover, in this preferred embodiment, the plug 24 is further configured to reseal the at least one opening 21 of the two-way valve system 20 when the actuator 26 stops being actuated. Therefore, after the actuator 26 is actuated, the at least one opening 21 is resealed by the plug 24. For example, in the embodiment of FIG. 1, when the actuator 26 stops being actuated the plug 24 is shaped in a spring-like pattern that when made of an elastic material, after being compressed during the displacement by the actuator 26, recovers its original position, therefore resealing the at least one opening 21 of the two-way valve system 20. Advantageously, this configures the two-way valve 20 to be configured to passively remain closed whenever the actuator 26 is not actuated. This means that the stoma plug is configured to be closed and sealed whenever the valve it's not actuated, so it further prevents the flow of the biological fluids when in use, i.e. in the daily life of the user. and provides self-control for stool continence. Moreover, as the two-way valve system 20 can be closed again once opened, there is no need to change the anti-reflux cap every time the stool is removed, making it reusable. The plug 24 may comprise an elastic and or flexible material that can recover its original shape once an external force quits being applied into it. Therefore, the plug 24 may act as a spring element.

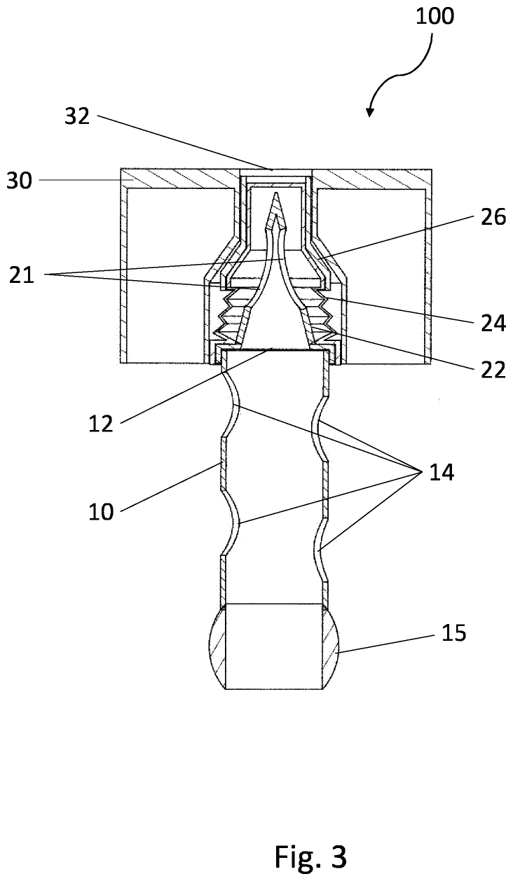

More preferably, the base element 22 is configured to perforate the plug 24 when the plug 24 is displaced by the actuator 26. FIG. 3 shows a schematic cross-sectional view of a stoma plug 100 according to at least one of the embodiments of the invention. It is noted that the stoma plug 100 of FIG. 3 comprises a tube 10, a two-way valve system 20 and a cap 30. However, it further comprises the optional elements: several openings 14 in the distal portion of the tube 10, a rounded tip 15 on the distal portion of the tube 10, comprises two openings 21. However, in other embodiments, there may be one or different number of openings 14 in the distal portion of the tube 10, may be no rounded tip 15 on the distal portion of the tube 10 and one or more than two openings 21. It is noted that the base element 22 may perforate the plug in several ways. The person skilled in the art may devise different ways in which the base element 22 is configured to perforate the plug 24 when the plug 24 is displaced by the actuator 26. For example, the base element 22 may comprise a perforating element that is actuated by the actuator 26 when the actuator is actuated, so that when the plug 24 is displaced towards the base element 22, the perforating element is also actuated and perforates the plug 24.

It can be seen that in FIG. 3 the plug 24 is configured to seal the at least one opening 21 of the base element 22, and that the actuator 26 is configured to longitudinally displace the plug 24 towards the base element 22 to unseal the at least one opening 21. However, it can be noticed that when the plug 24 is displaced by the actuator 26, it does not allow to unseal the element unless the base element 22 perforates the plug 24, therefore generating an aperture from wherein the fluids can flow.

Advantageously this configures the plug 24 to be pre-sealed before the first use, therefore providing an additional security measure against biological fluids from the stoma to leak to the outside when in use until the first aperture of the two-way valve system through the actuation of the actuator. Once the plug 24 is perforated by the base element 22 and after the actuator 26 stops being actuated, the plug 24 reseals the opening 21, but the plug 24 remains perforated. It is noted that the opening 21 seal is not compromised by the perforation of the plug 24.

In an even more preferred embodiment, the base element 22 has a spiky shape, more preferably wherein the spiky shape is essentially of a cone shape. Advantageously, this configures the base element 22 to be able to perforate the plug 24, when the plug 24 is displaced towards the base element 22 by the actuator 26. Therefore, the plug 24 displacement by the actuator 26 is the one that causes the base element 22 to perforate the plug 24.

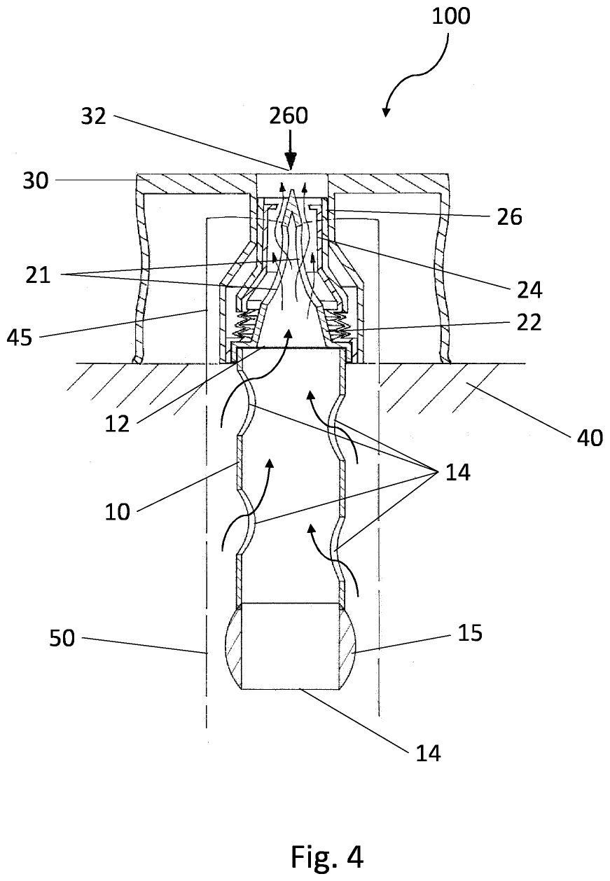

FIG. 4 shows the schematic cross-sectional view of an actuated stoma plug 100 inserted in a stoma 50, according to at least one of the embodiments of the invention. It is noted that the stoma plug 100 of FIG. 4 comprises a tube 10, a two-way valve system 20 and a cap 30. However, it further comprises the optional elements: several openings 14 in the distal portion of the tube 10, a rounded tip 15 on the distal portion of the tube 10, comprises two openings 21. However, in other embodiments, there may be one or different number of openings 14 in the distal portion of the tube 10, may be no rounded tip 15 on the distal portion of the tube 10 and one or more than two openings 21. Moreover, it's noted that the stoma plug 100 is inserted into a stoma 40 and that the cap 30 is configured to cover the two-way valve system 20 and the stoma external mucosa 45, and adapted to the skin surface 40. It is noted that the cap 30 may take several shapes to adapt to the skin surface 40.

The stoma plug 100 of FIG. 4 is actuated as shown by arrow 260 through the opening 32 of the cap 30, such that the actuator 26 has displaced the plug 24 and unsealed the at least one opening 21 of base element 22. Moreover, the base element 22, has a spiky shape in form of a cone, that has perforated the plug 24. In this configuration, the two-way valve-system 20 has the plug 24 displaced from the at least one opening 21, therefore fluidly connecting the biological fluids of the colon, ileum and/or ureter 50 (such as the stool) through the openings 14 at the distal portion of the tube 10, which in turn provides access to the openings 21 of the base element 22 through the opening 12 at the proximal portion of the tube 10. Moreover, as the actuator 26 has been actuated (as shown by arrow 260), the plug 24 has unsealed the openings 21 and the biological fluids of the colon, ileum and/or ureter 50 can reach the exterior through the opening 32 of the cap 30. Therefore, when the actuator 26 is actuated, the biological fluids of the stoma 50 are fluidly connected to the exterior of the body.

It is noted that the two-way valve system 20, allows for an external fluid to be inserted into the colon, ileum and/or ureter 50 through the stoma 45 when the actuator 26 is actuated, as it is fluidly connected to the interior of the colon, ileum and/or ureter 50. For example, a mixture of lactulose and water or lactulose and cola drink may be introduced into the colon, ileum and/or ureter 50 through the stoma 45 to fight or prevent an intestinal obstruction.

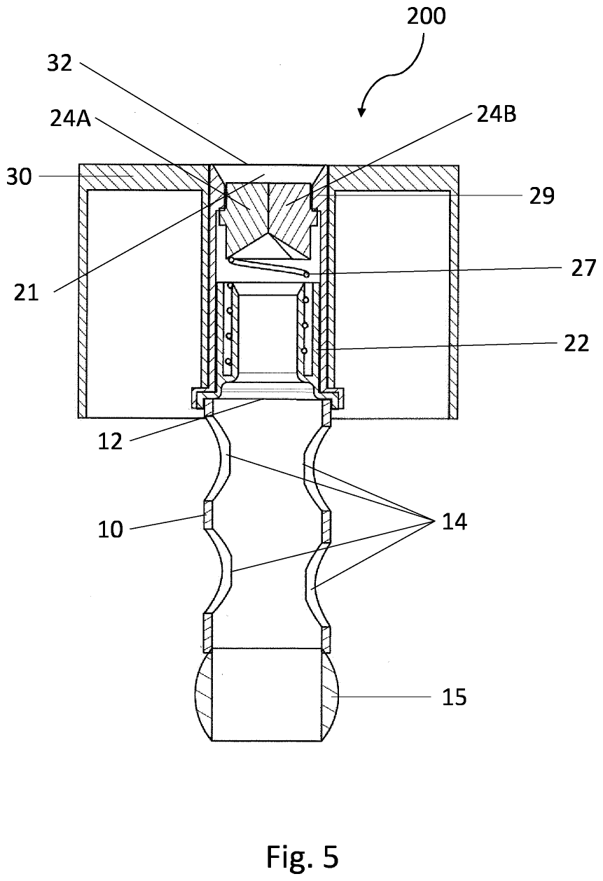

Alternatively, in another preferred embodiment, as shown in FIG. 5, the two-way valve system further comprises: a spring 27, a base element 22 configured to be slidably receivable by the spring 27, and a plug stopper 29 comprising the at least one opening 21 of the two-way valve system 20. It is noted that the stoma plug 200 of FIG. 5 comprises a tube 10, a two-way valve system 20 and a cap 30. However, it further comprises the optional elements: several openings 14 in the distal portion of the tube 10, a rounded tip 15 on the distal portion of the tube 10, and a two-piece plug 24. However, in other embodiments, there may be one or different number of openings 14 in the distal portion of the tube 10, may be no rounded tip 15 on the distal portion of the tube 10 and the plug 24 may not comprise more than one piece or comprise several pieces. In some embodiments, as in FIG. 5, the base element 22 is a fixed element of the two-way valve system, so that when the spring is compressed, it is not displaced. The base element 22 holds the spring into a particular direction so that the compression direction is constant. The base element 22 is further the element of the two-way valve system 20 through which the two-way valve system 20 is connected to the proximal portion of tube 10.

The spring 27 is configured to press the plug 24 against the plug stopper 29 and close the at least one opening 21 of the two-way valve system 20. It is noted that the person skilled in the art may envisage several ways in which a spring 27 can press a plug 24 against a plug stopper 29 to close the at least one opening 21 of the two-way valve system 20. For example, the spring 27 may be a compression spring and be at least partially compressed so that it exerts an extension force. If the plug 24 is on one side of the spring 27 and the base element 22 is on the other side as a support, when the plug 24 is oriented towards the plug stopper, the spring 27 presses the plug 24 against the plug stopper 29 and close the at least one opening 21 of the two-way valve system 20. Alternatively, the spring 27 may be an extension spring and be at least partially stretched so that it exerts a compression force. If the plug 24 is attached to one side of the spring 27 and the base element 22 is on the other side as a support, when the plug stopper 29 is between the spring 27 and the base element 22, the spring 27 presses the plug 24 against the plug stopper 29 and close the at least one opening 21 of the two-way valve system 20. Equivalent systems may be designed by the person skilled in the art using one or more torsion springs, drawbar springs, volute springs, and any other type of spring as known in the art. In FIG. 5, the spring 27 is a compression spring, that presses the plug 24 against the plug stopper 29, therefore closing the at least one opening 21.

Moreover, the spring 27 is further configured to longitudinally displace from the plug stopper 29 when a force is applied, thus displacing the plug 24 from the at least one opening 21 and therefore opening the at least one opening 21 of the two-way valve system 20. It is noted that the person skilled in the art may envisage several ways in which a spring 27 can be displaced from the plug stopper 29 when a force is applied. Several options may be defined according to the type of spring as defined above. For example, in FIG. 5 the compression spring 27 can be compressed, and as the base element 22 is fixed, the plug 24 is thus displaced from the at least one opening 21 of the two-way valve system 20, leading to the opening of the at least one opening 21.

Advantageously, having a spring-actuated two-way valve system 20 allows for a reliable opening and closing mechanism, that works in a positive feedback to reinforce the sealing of the at least one opening 21 of the two-way valve system 20. For example, in the embodiment as shown in FIG. 5, if the pressure of the biological fluids of the stoma increases, the pressure further presses the plug 24 against the plug stopper 29 and further closes the at least one opening 21 of the two-way valve system 20. By this way the stoma plug is configured to further prevent the involuntary flow of the biological fluids when in use.

In a further preferred embodiment, as shown in FIG. 5, the plug 24 comprises two pieces 24A, 24B. The two pieces 24A, 24B are configured to remain in contact when the spring 27 presses them against the plug stopper 29 and to separate apart when the spring 27 is longitudinally displaced, thus generating an aperture between the two pieces 24A, 24B and allowing the at least one opening 21 of the two-way valve system 20 to be accessible through the aperture generated between the two pieces 24A, 24B.

The two pieces 24A, 24B may be two equal pieces or may be pieces that have different shapes. Altogether, the two pieces 24A, 24B when in contact they fully close the at least one opening 21 of the two-way valve system 20. The person skilled in the art may note this can be done in several ways. For example, one of the pieces 24A may be a ring and the other one 24B be a plug closing the inner circle left by the ring of the first piece 24A. In the example of FIG. 5, the two pieces 24A, 24B are pieces each covering half of the circumference left by the at least one opening 21. Therefore, both pieces 24A, 24B have the same shape. However, in other embodiments these may have different shapes. It is noted, that in the embodiments when there are more than one opening 21, each of the two pieces 24A, 24B may close one or more than one of the opening 21.

The two pieces 24A, 24B may be configured to separate apart when the spring 27 is longitudinally displaced in several ways. For example, each of the two pieces 24A, 24B may have a lever or a ramp that interacts with the spring 27 or any of the surrounding elements such as the base element 22, so that the two pieces 24A, 24B move in opposite directions outwardly, therefore generating an aperture between the two pieces 24A, 24B. Through this aperture, the at least one opening 21 of the two-way valve system 20 can be accessed from the interior, i.e. the plug 24 is no longer closing the at least one opening 21. Consequently, as the at least one opening 21 is fluidly connected to the biological fluids in the tube distal portion through the opening 12 in the proximal portion of the tube 10 and the at least one opening 14 in the distal portion of the tube 10, the biological fluids at the distal portion of the tube 10 are fluidly communicated with the exterior.

In use, the two-way valve system 20 of the embodiment according to FIG. 5 is configured to be opened when the spring 27 is longitudinally displaced towards the base element 22, i.e. by compressing the spring. This makes the two pieces 24A, 24B to separate apart, as each have a ramp shaped base that interacts with the base element 22 and displaces them outwardly, allowing the biological fluids from the colon, ileum and/or ureter 50 to be fluidly connected to the at least one opening 21 of the two-way valve system 20 through the aperture generated between the two pieces 24A, 24B.

Advantageously, having the at least one opening 21 of the two-way valve system 20 accessible through the aperture generated between the two pieces 24A, 24B allows for a better flow through the at least one opening 21, as the aperture generated between the two pieces 24A, 24B is aligned with the at least one opening 21. Having these aligned, reduces the turbidity of the fluid flow which can reduce the probability of clogging the two-way valve system by any small solid present in the fluid.

It is noted that in this alternative preferred embodiment, the spring 27 is configured to longitudinally displace from the plug stopper 29 when a force is applied. However, this force may be applied by the user at a will or using any external element through the opening 32 of the cap 30.

In a preferred embodiment of any of the previous embodiments, the tube 10 comprises a support element, configured to receive the base element 22 of the two-way valve system 20. Also, the two-way valve system 20 is comprised within the tube 10. It is noted that the two-way valve system 20 may be comprised within the tube 10 entirely or just partially, as long as the two-way valve system 20 can be directly accessed through the opening 32 of the cap 30.

Advantageously, this preferred embodiment allows for a much compact design of the stoma plug, as the two-way valve system 20 can be at least partially hosted by the tube 10, and therefore the cap 30 can be diminished, allowing for a slimmer design of the cap 30, further increasing the self-esteem for carriers.

In a preferred embodiment of any of the previous embodiments, the plug 24 comprises a spring-like section, configured to help the plug to reseal the at least one opening 21 of the two-way valve system 20 when the actuator 26 stops being actuated. The spring-like section may comprise a metallic material. In those embodiments wherein the base element 22 is configured to perforate the plug 24 when the plug 24 is displaced by the actuator 26, in order to facilitate the plug 24 to comprise both a surface to be perforated, and a spring-like section, the surface to be perforated may be located in the proximal portion of the plug, and the spring-like section may be located in the distal portion of the plug 24. The surface to be perforated and the spring-like portion may be connected through a frame.

In a preferred embodiment of any of the previous embodiments, the at least one opening 14 of the distal portion of the tube 10 is at least laterally placed on the tube 10. As shown in FIGS. 1-5 some of the openings 14 in the tube 10 distal portion are laterally placed. However, in other embodiments, there may be an opening 14 on the tip but partially laterally placed, or when the stoma plug comprises only one opening 14 in the tube 10 distal portion, the opening may be only lateral or at least laterally placed in the tube 10, so that part of the opening is at the tip and part on the lateral of the tube 10. Advantageously, this allows for the biological fluids in the colon, ileum and/or ureter 50 to keep flowing into the tube 10 through the at least one opening 14, whenever a big piece of stool is placed at the tip of the tube 10. As the stool exercises a pressure at the colon, ileum and/or ureter 50 end due to the peristaltic movement at the point, the at least one opening 14 at the tip of the tube 10 at the distal portion may be blocked, but the at least laterally placed portion of the at least one opening 14, is kept clear from the stool, while the biological fluids can keep reaching such lateral at least one opening 14 at least laterally placed on the tube 10.

In another preferred embodiment of any of the previous embodiments, the at least one opening on the distal portion 14 of the tube 10 is smaller than 15 mm. The present stoma plug is preferably designed for the control of liquids such that of ileostomies and urostomies, as well as some colostomies. Therefore, the at least one opening 14 on the distal portion of the tube 10 may be used to control the size of the elements that reaches the two-way valve system 20. Advantageously, having the at least one opening 14 on the distal portion of the tube 10 smaller than 15 mm configures the stoma plug to prevent any element such as stool bigger than 15 mm to be extracted when the two-way valve system 20 is opened, which could clog the two-way valve system 20. It is noted that in FIGS. 1-5, the at least one opening 14 are circles, but that in other embodiments, these one opening may take different shapes as the person skilled in the art may foresee such as ovals, squares or any other shape. In the case of the FIGS. 1-5 the size of the at least one opening 14 is determined by the diameter of the openings, while in other embodiments, the size of the at least one opening 14 is defined by the maximum diameter of the opening 14 in any direction over the surface of the tube 10. In a more preferred embodiment, the at least one opening 14 on the distal portion of the tube 10 is smaller than 10 mm.

In another preferred embodiment of any of the previous embodiments, the tube 10 comprises a rounded tip 15 on its distal portion, configured to facilitate the insertion of the tube 10 into the stoma 50. As the tube 10 is configured to be introduced into a colon, ileum and/or ureter 50, having a rounded tip 15, further facilitates the insertion of the tube through the stoma 45, such that the internal mucosa of the colon, ileum and/or ureter 50 can slide as the tube 10 is introduced through the stoma 45 into the colon, ileum and/or ureter 50. Therefore, this feature further allows for a painless insertion of the stoma plug.

In a more preferred embodiment of the previous embodiment, the rounded tip 15 on the distal portion of the tube 10 has a rounded cone shape. Advantageously, a rounded-cone shape tip 15 not only facilitates the insertion of the tube 10 into the stoma 45, but it further increases the contact surface between the tube 10 and the internal mucosa of the colon, ileum and/or ureter 50. The term “rounded cone” is preferably understood as a cone whose tip has been rounded. It is noted that in the context of the present invention, a tip with a rounded cone shape is a tip wherein the tip of the cone is in the distal end of the tip. The skilled person may envisage many different alternatives of tips shaped like a rounded cone with different widths and cone tip radius, all of them comprised within this particular embodiment.

In another preferred embodiment of any of the previous embodiments, the tube 10 external surface further comprises at least a spiral portion configured to increase the contact surface of the tube 10 with the colon, ileum and/or ureter 50. Having at least a spiral portion on the externals surface of the tube, generates a rougher surface to which mucosa of the colon, ileum and/or ureter 50 can attach to. Advantageously, this provides a better grip between the internal mucosa of the colon, ileum and/or ureter 50 and the tube 10, providing a more secure attachment of the stoma plug to the colon, ileum and/or ureter 50. It is noted that many spiral configurations may be envisaged. For example, the spiral may be outwardly radially extending from the external surface of the tube 10, or it may be inwardly radially extending into the external surface of the tube 10. In some embodiments, the spiral may have different degrees of turn per length of the tube 10 and may turn in one or another sense (i.e. clockwise or counterclockwise). It may also turn in one sense in one portion of the tube 10 and in another sense in another portion of the tube 10.

In another preferred embodiment of any of the previous embodiments, the tube 10 is comprised of any of the materials selected from: medical grade silicone, surgical plastic, thermoplastic elastomers, or any other biocompatible material including biocompatible polymers. Advantageously, this configures the tube 10 to be inserted through the stoma 45 into the colon, ileum and/or ureter 50 to be biocompatible and at least partially flexible so that it can adapt to the colon, ileum and/or ureter 50 shape. This means that in use, the tube 10 can bend when the colon, ileum and/or ureter 50 bends therefore reducing the pain and increasing the self-esteem of the user.

In another preferred embodiment of any of the previous embodiments, the tube 10 further comprises a lubricated layer on its external surface, configured to reduce the friction during its insertion through the stoma 45. This layer may comprise any antifriction substance such as oily substances or water-based lubricants, and may further comprise anaesthetic components, such as tetracaine or lidocaine. Advantageously, this configures the tube 10 to be inserted through the stoma 45 without friction and therefore increasing the ease of use and reducing the pain felt by the user.

In another preferred embodiment of any of the previous embodiments, the two-way valve system 20 further comprises a heat-sensitive hydrocolloid surface configured to stick or adapt to the stoma 45. As the colon, ileum and/or ureter 50 biological fluids may be at a higher pressure inside the stoma than the atmosphere pressure, it is desired to have the stoma plug 100, firmly attached to the stoma 45. As shown for example with respect to FIG. 4 the two-way valve system 20 interacts with the stoma 45, to fix the stoma plug 100 into the colon, ileum and/or ureter 50. Therefore, having a hydrocolloid surface that is absorbent, self-adhesive, and waterproof allows for the stoma plug 100 to be firmly affixed to the stoma. Moreover, a heat-sensitive surface may only adhere to heated surfaces such as the stoma, making the stoma plug 100 easy to manipulate before the insertion into the colon, ileum and/or ureter 50. Advantageously, having the two-way valve system 20 further comprises a heat-sensitive hydrocolloid surface configured to stick to the external stoma mucosa 45 configures the stoma plug 100, 200 to be kept in place when the internal pressure in the colon, ileum and/or ureter 50 increases over the atmosphere pressure.

In a more preferred embodiment, the heat-sensitive hydrocolloid surface comprises a dressing configured to adhere to the subject's skin 40 extending radially from the two-way valve system 20. Advantageously, this further increases the adherence of the stoma plug 100, 200, by further adhering to the user's skin. It is noted that the dressing may extend radially with different lengths in each direction and that the shape may be adapted to the location of the stoma 45 and/or to the size of the stoma 45. In an even more preferred embodiment the dressing is transparent, providing a less visible design and further increasing the self-esteem of the subject.

In another preferred embodiment of any of the previous embodiments, the two-way valve system 20 further comprises an adaptative biocompatible material configured to stick or adapt to the stoma 45. The adaptative biocompatible material is configured to adapt to the stoma external mucosa and the peristomal skin surface, such that a tighter affix between the stoma plug and the stoma is achieved.

In another preferred embodiment, the stoma plug 100, 200 further comprises a pressure sensor to monitor the pressure inside the colon, ileum and/or ureter 50. The two-way valve system 20 may then be opened automatically, when the pressure sensor detects that a certain pressure threshold is reached or it may indicate the user through a feedback element that the two-way valve system 20 shall be opened to drain the biological fluids of the colon, ileum and/or ureter 50.

A second aspect of the invention refers to a stoma plug system comprising a stoma plug 100, 200 according to any of the stoma plugs of the first aspect of the invention and a collecting bag or a vacuum device. The collecting bag or vacuum device is configured to connect to the two-way valve system 20 and to open the at least one opening 21 displacing the plug 24 from the at least one opening 21. Preferably, the collecting bag or vacuum device is configured to connect to the two-way valve system 20 through the opening 32 of the cap 30.

Therefore, when the collecting bag or the vacuum device is connected to the two-way valve system 20, the stoma plug is open, and the biological fluids of the colon, ileum and/or ureter 50 can get to the exterior through the stoma plug. Both the collecting bag and the vacuum device are thus configured to deal with the biological fluids that may come out form the stoma plug. The collecting bag can be any type of collecting bag as known in the stoma device industry. Thus, the collecting bag may be a pouch opened at the bottom so that the contents collected can be disposed through the opening on the bottom, or it may be a pouch closed at the bottom and it may therefore be replaced when the contents need to be disposed. The vacuum device therefore either comprises a collecting recipient to store the biological fluids or is connected to a recipient where these can be stored.

It is noted that while the collecting bag collects the biological fluids of the colon, ileum and/or ureter 50 that passively exits the stoma plug, the vacuum device actively extracts the biological fluids of the colon, ileum and/or ureter-50 by using a negative pressure when compared to the internal pressure of the colon, ileum and/or ureter 50.

The collecting bag or vacuum device opens the at least one opening 21 displacing the plug 24 from the at least one opening 21 in several ways. For example, it may open the at least one opening 21 by directly displacing the plug 24 from the at least one opening 21 when it is connected to the two-way valve system 20, or it may do so by actuating on an actuator 26 at the moment it is when it is connected to the two-way valve system 20. Alternatively, the collecting bag or vacuum device may comprise a second mechanism on the connection element to the at least one opening 21 of the two-way valve system 20 that may be manually or electrically actuated to open the two-way valve system 20.

In the embodiments, wherein the stoma plug 100, 200 comprises a pressure sensor to monitor the pressure inside the colon, ileum and/or ureter 50, the sensor may send a signal to the user indicating when is the adequate moment to drain the biological fluids of the colon, ileum and/or ureter 50 by using the collecting bag or a vacuum device.

Advantageously, a stoma plug system comprising a stoma plug 100, 200 according to any of the stoma plugs of the first aspect of the invention and a collecting bag or a vacuum device allows the user to either allow the biological fluids of the colon, ileum and/or ureter 50 to passively be drained by using a collecting bag, or to manually and actively draining the biological fluids of the colon, ileum and/or ureter 50 using the vacuum provided by the vacuum device.

In a preferred embodiment, the vacuum device is a syringe. Advantageously, the user can use the syringe to extract in a short period of time the biological fluids of the colon, ileum and/or ureter 50 through the stoma plug. Therefore, the user can easily release the biological fluids of the colon, ileum and/or ureter 50 through the stoma 45 in fast and clean way. The syringe may then be emptied in a toilet. The syringe may be reusable or disposable.

1. A stoma plug suitable for controlling the output of biological fluids from a colon, ileum and/or ureter through a stoma created by a colostomy, ileostomy or urostomy respectively, the stoma plug comprising:
a) a tube comprising an opening in a proximal portion and at least one opening in a distal portion,
b) a two-way valve system comprising a base element and a plug, the two-way valve system connected to the tube through the base element, the two-way valve system configured to control the fluid flow through the opening of the proximal portion of the tube, and
c) a cap configured to cover the two-way valve system and the stoma, comprising an opening to directly access the two-way valve system from the outside,
wherein the tube is configured to be inserted into the colon, ileum and/or ureter through its distal portion, and
wherein the two-way valve system is selected from one of the following two alternatives:
i. the two-way valve system further comprises an actuator, the base element comprises at least one opening, wherein the two-way valve system is configured to passively remain closed such that when the actuator is not actuated the plug is configured to cover the base element and seal the at least one opening; and is further configured to be opened when the plug is displaced from the at least one opening such that the actuator is configured to displace the plug and unseal the at least one opening of the two-way valve system when actuated and wherein the plug is further configured to reseal the at least one opening of the two-way valve system when the actuator stops being actuated; or
ii. the two-way valve system further comprises a spring and a plug stopper comprising at least one opening, the base element configured to be slidably receivable by the spring, wherein the two-way valve system is configured to passively remain closed such that the spring is configured to press the plug against the plug stopper and close the at least one opening of the two-way valve system and is further configured to be opened when the spring is longitudinally displaced from the plug stopper when a force is applied, thus displacing the plug from the at least one opening and therefore opening the at least one opening of the two-way valve system.

2. The stoma plug according to claim 1, wherein the two-way valve system further comprises an actuator, the base element comprises at least one opening, wherein the two-way valve system is configured to passively remain closed such that when the actuator is not actuated the plug is configured to cover the base element and seal the at least one opening; and is further configured to be opened when the plug is displaced from the at least one opening such that the actuator is configured to displace the plug and unseal the at least one opening of the two-way valve system when actuated and wherein the plug is further configured to reseal the at least one opening of the two-way valve system when the actuator stops being actuated.

3. The stoma plug according to claim 1, wherein the two-way valve system further comprises a spring and a plug stopper comprising at least one opening, the base element configured to be slidably receivable by the spring, wherein the two-way valve system is configured to passively remain closed such that the spring is configured to press the plug against the plug stopper and close the at least one opening of the two-way valve system and is further configured to be opened when the spring is longitudinally displaced from the plug stopper when a force is applied, thus displacing the plug from the at least one opening and therefore opening the at least one opening of the two-way valve system.

4. The stoma plug according to claim 2, wherein the base element is configured to perforate the plug when the plug is displaced by the actuator.

5. The stoma plug according to claim 2, wherein the base element has a spiky shape, more preferably wherein the spiky shape is essentially of a cone shape.

6. The stoma plug according to claim 3, wherein the plug comprises two pieces, and wherein the two pieces are configured to remain in contact when the spring presses them against the plug stopper and to separate apart when the spring is longitudinally displaced, thus generating an aperture between the two pieces and allowing the at least one opening of the two-way valve system to be accessible through the aperture generated between the two pieces.

7. The stoma plug according to claim 1, wherein the tube comprises a support element configured to receive the base element of the two-way valve system, and wherein the two-way valve system is comprised within the tube.

8. The stoma plug according to claim 1, wherein the at least one opening on the distal portion of the tube is smaller than 15 mm.

9. The stoma plug according to claim 1, wherein the tube comprises a rounded tip on its distal portion, configured to facilitate the insertion of the tube through the stoma.

10. The stoma plug according to claim 9, wherein the rounded tip on the distal portion of the tube has a rounded cone shape.

11. The stoma plug according to claim 1, wherein the tube external surface further comprises at least a spiral portion configured to increase the contact surface of the tube with the inner surface of the colon, ileum and/or ureter.

12. The stoma plug according to claim 1, wherein the tube is comprised of any of the materials selected from: medical grade silicone, surgical plastic thermoplastic elastomers or any biocompatible material.

13. The stoma plug according to claim 1, wherein the tube further comprises a lubricated layer on its external surface, configured to reduce the friction with the stoma during its insertion.

14. The stoma plug according to claim 1, wherein the two-way valve system further comprises a heat-sensitive hydrocolloid surface or an adaptative biocompatible material surface configured to stick or adapt to the external stoma mucosa.

15. The stoma plug according to claim 14, wherein the heat-sensitive hydrocolloid or the adaptative biocompatible material surface comprises a dressing configured to adhere to the subject's skin extending radially from the two-way valve system, preferably wherein the dressing is transparent.

16. The stoma plug according to claim 1, wherein the at least one opening of the distal portion of the tube is at least laterally placed on the tube.

17. A stoma plug system, comprising:
a) a stoma plug according to claim 1, and
b) a collecting bag or a vacuum device;
wherein the collecting bag or vacuum device is configured to connect to the two-way valve system and to open the at least one opening by displacing the plug from the at least one opening more preferably, wherein the collecting bag or vacuum device opens the at least one opening through its connection to the two-way valve system.

18. The stoma system according to claim 17, wherein the vacuum device is a syringe.
